(19) United States
(12) Patent Application Publication
(10) Pub. No.: US 2012/0059403 A1
(43) Pub. Date: Mar. 8, 2012

(54) VENOUS DISSECTOR DEVICE

(76) Inventor: Jose Eduardo Domingues, Sao Jose dos Campos / SP (BR)

(21) Appl. No.: 13/223,440

(22) Filed: Sep. 1, 2011

(30) Foreign Application Priority Data
Sep. 3, 2010 (BR) PI 1003540-0

(51) Int. Cl. A61B 17/00

(52) U.S. Cl. 606/190

(57) ABSTRACT

Venous dissector device, notably a seamless device (1), preferably made in polymer, comprising a ergonomic rod (2), followed by a central region (3) substantially elliptical, with grooves (4) for a better handling, and a tip (5) with end hook (6), responsible for exposing the sick vein.

### CROSS-REFERENCE TO RELATED APPLICATIONS

This application claims priority under 35 U.S.C. §119 to Brazilian Application PI 1003540-0 filed Sep. 3, 2010, herein incorporated by reference in its entirety.

### BACKGROUND OF THE INVENTION

This patent of invention request deals with an unpublished venous dissector device, notably a disposable device designed specifically for use in the surgical procedures of essential varicose veins in the lower limbs, well known as peripheral vascular surgery, which feature is to format an unique instrument with a hook shaped end with suitable size for this kind of intervention, making the procedures simpler, safer and more accurate.

Basically, varicose veins are veins that dilate for several reasons. Varicose veins may appear of several ways, from small ones to those that forms nodules that appears through the skin. They are more common in women but also can appear, at a lower rate, in men. In short, varicose veins affect blood circulation, causing pain and fatigue in the legs. For each type of varicose veins there is an appropriate way of treatment that may be clinical or surgical.

Regarding to the peripheral vascular surgery, the current prior art shows a complete stagnation. The procedures are made, for many years, by small incisions in the skin, made by cold scalpel on the affected region. The incisions provide the useful exposition, out of the skin, of the varicose veins, where they are captured, sectioned and their parts removed, until the complete removal of the varicose veins.

When exposed, sick veins are dissected by a rudimentary method, with aid of a non-surgical device, popularly named “knitting needle”.

Kafejian et al. has published their work in 1976, where the knitting needle was used to externalize varicose veins by tiny incisions.

The laser technique is certainly effective; however, it is indicated for removal of the saphenous vein and not the resection of collateral varicose veins.

Despite being widely used, the knitting needle used in the procedures is a rough adaptation, not consistent with the hospital environment, presenting practical limitation and potential damages. One of the most prominent limitations relates to the reduced dimensional variety of such needles, so that very small veins are inaccessible and inoperable.

On the other hand, because it is an instrument made of a regular metal alloy, the knitting needle is susceptible to wear of the material that may result in deformation or breakage of the instrument by use, fragmentation of the surface into small parts, which causes uniformity loss, rust and finally accumulation of waste and impurities of previous procedures, potentially infecting, housed at the needle tip as, for instance, blood, vein fragments, fragments of subcutaneous tissues, etc. Add to these negative aspects, the fact that the alloy is totally unsuitable for use in autoclaves, ovens and sterilization devices of common use in hospitals and clinics, which causes progressive degradation of each cleaning, since it is not a disposable instrument.

Other alternative instruments are also known, as such Petean forceps, more efficient for larger incisions.

Prior art anticipates patent documents related to devices of delamination or dissection for purposes others than peripheral vascular surgery, as PI0511315-6 “Epithelial Delamination Device”, which refers to a device used in the field of ophthalmology. Device and methods of use include separating or lifting the corneal epithelium of eye in a substantially continuous layer to form a flap or pouch.

MU7601078-3 “Spherical Dissector with Thermo Cautery” refers to a surgical instrument that, due to its angled arrangement with a metallic sphere at its end, facilitates the recognition and dissection of an easy and safe way of the ductal and vascular structures next to the gallbladder.

### SUMMARY OF THE INVENTION

Aware of the prior art, its gaps and limitations, the inventor, a person acquainted to this matter, after studies and analyses, created the venous dissector device in subject, which is a disposable surgical instrument, preferably made of polymer, whose constructiveness makes it suitable for dissection of varicose veins of all sizes and calibers, since the end hook has a dimensional variety that reaches all range of said veins, empirically checked, in which the dimensions are correlated with color differences.

The invented device presents as main advantages:
Designed specifically for hospital use, in procedures for varicose veins in the lower limbs;
Disposable, avoiding infections and other complications recurring from several sterilizations common to known models;
Lighter, for being a polymer;
Resistant to breakage and deformation;
Not subjected to fragmentation of surface;
Anatomic and ergonomic, providing better maneuverability for the professional;
More precise surgical handlings;
Accurate hooks and fewer exposure attempts of the veins, ensuring faster and safer procedures, with less aggressiveness to the veins;
Dimensional identification (Frenchs) by colors (standardization recognized by the competent authorities), which facilitates the fast visualization during the procedure.
Great for use in the veins of small caliber;

Therefore, the device makes the intervention in varicose veins simpler, safer and more accurate, avoiding inconveniences to the patient and the professional.

To follow, the invention is explained with reference to the attached drawings, in which they are represented, in an illustrative and not limiting way:

### DESCRIPTION OF THE FIGURES

FIG. 1 shows a perspective view of venous dissector device;

FIG. 2 shows an upper view of venous dissector device;

FIG. 3 shows a side view of venous dissector device;

FIG. 4 shows a detail of the central handle area of venous dissector device;

FIG. 5 shows a side view of venous dissector device in another embodiment, with a thicker rod.

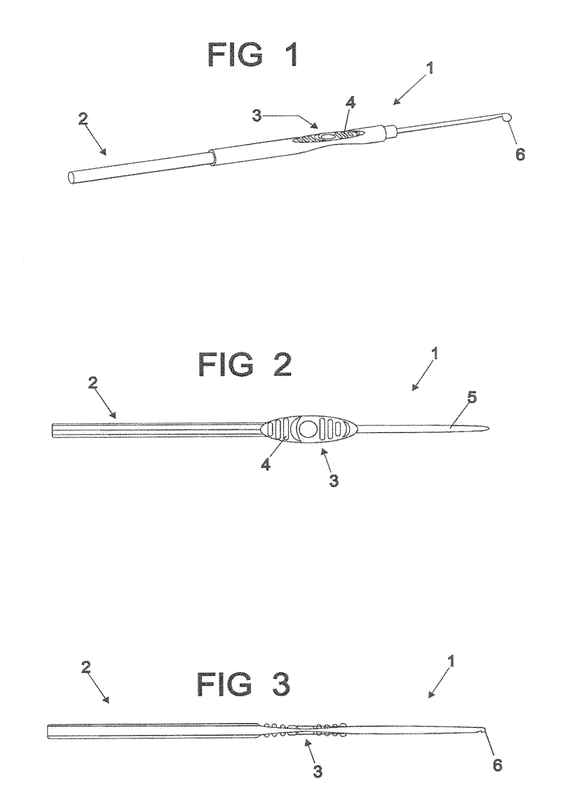

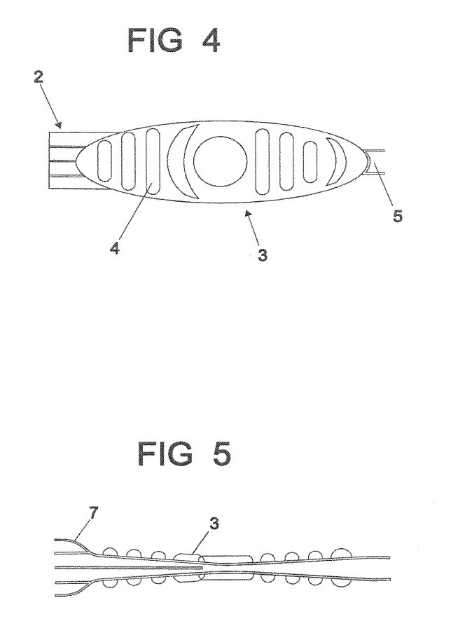

### DESCRIPTION OF THE PREFERRED ASSEMBLY OF THE INVENTION

The venous dissector device of this invention consists essentially of a seamless device (1), preferable made of polymer, comprising an ergonomic rod (2), followed by a central region (3), substantially elliptical, with grooves (4) for a better handling, and a tip (5) with end hook (6), responsible for exposing the sick vein.

More particularly, the seamless device (1) is designed for varicose veins surgery and made of solid polymer or with a polymer with metallic soul, which has a massive rod (2) of octagonal section that follows a regular plane until the central region (3) of contact between the professional fingers and said device (1). For this, such region presents an elliptical design with grooved surface finish (4), in order to improve/increase the grip and therefore the safety during the procedure. From said central region (3) derivates a tip (5), which forms a hook (6) suitable for exposing the altered vein. The device (1) will be provided with hooks (6) of dimensional strips appropriate to the sizes of the veins, empirically checked, and each strip will correspond to certain color standardized by the competent authorities.

In another embodiment, the rod (2) of device is thicker, which requires an angled approach (7) toward the central region (3).

What is claimed is:

1. Venous dissector device, wherein said device is seamless, made of solid polymer or with a polymer with metallic soul, which has a massive rod (2) of octagonal section that follows a regular plane until the central region (3), of elliptical design with grooved surface finish (4), from where derivates a tip (5), which forms a hook (6) suitable for exposing the altered vein.

2. Venous dissector device, according to claim 1, wherein said device presents a thicker rod (2), which requires an angled approach (7) toward the central region (3).
